(19) United States
(12) Patent Application Publication
(10) Pub. No.: US 2019/0159926 A1
(43) Pub. Date: May 30, 2019

(54) WAIST SUPPORTED OSTOMY APPLIANCE CASE

(71) Applicant: Estie Braff, Chagrin Falls, OH (US)

(72) Inventor: Estie Braff, Chagrin Falls, OH (US)

(21) Appl. No.: 15/822,430

(22) Filed: Nov. 27, 2017

(51) Int. Cl. A61F 5/449; A61F 5/448

(52) U.S. Cl. CPC A61F 5/449; A61F 5/448

(57) ABSTRACT

The present disclosure provides a waist supported ostomy appliance case that effectively and comfortably conceals an ostomy appliance. The case includes a belt for securing the case to the waist of an individual and a bag positioned on the belt for enclosing the ostomy appliance. A rear panel of the bag includes a stoma opening configured to fit around a flange of the ostomy appliance. A bag bottom of the bag includes a bottom recloseable opening into a bag interior. The bottom recloseable opening provides access to a bottom portion of the ostomy appliance for emptying the ostomy appliance without the need to remove the case. The case may include one or more exterior pockets for storing personal belongings.

### FIELD OF INVENTION

The present disclosure relates generally to a case for an ostomy appliance, more particularly to a waist supported ostomy appliance case having a stoma opening and a bottom recloseable opening providing access to a bottom portion of the ostomy appliance.

### BACKGROUND

An ostomy is a surgical procedure which creates an artificial opening, or stoma, in the abdomen which is connected to the intestinal tract or the urinary tract for the discharge of bodily waist. People who have had ostomy surgery generally wear a bag or pouch connected to the stoma to collect fecal matter or urine discharged from the body.

### SUMMARY

The present disclosure provides a waist supported ostomy appliance case that effectively and comfortably conceals an ostomy appliance. The case includes a belt for securing the case to the waist of an individual and a bag positioned on the belt for enclosing the ostomy appliance. A rear panel of the bag includes a stoma opening configured to fit around a flange of the ostomy appliance. A bag bottom of the bag includes a bottom recloseable opening into a bag interior. The bottom recloseable opening provides access to a bottom portion of the ostomy appliance for emptying the ostomy appliance without the need to remove the case. The case may include one or more exterior pockets for storing personal belongings.

In accordance with one aspect of the present disclosure, a waist supported ostomy appliance case, includes: a belt for securing the case to the waist of an individual; and a bag positioned on the belt for enclosing an ostomy appliance, the bag including a bag interior defined between a rear panel, a front panel joined to the rear panel at opposite sides of the bag, a bag top, and a bag bottom, wherein the rear panel includes a stoma opening configured to fit around a flange of the ostomy appliance, and wherein the bag bottom includes a bottom recloseable opening into the bag interior, the bottom recloseable opening providing access to a bottom portion of the ostomy appliance.

In one embodiment, the belt includes a first belt member and a second belt member each attached to a corresponding one of the opposite sides of the bag.

In another embodiment, the first belt member and the second belt member include cooperating fasteners.

In another embodiment, at least one of the first belt member and the second belt member includes a locking clasp.

In another embodiment, the bag top is releasably secured to the rear panel by a fastener and hinges along a top edge of the bag to define a top recloseable opening into the bag interior providing access for insertion of the ostomy appliance into the bag interior.

In another embodiment, the bag top is releasably secured to the front panel by a fastener and hinges along a top edge of the bag to define a top recloseable opening into the bag interior providing access for insertion of the ostomy appliance into the bag interior.

In another embodiment, the bag bottom is releasably secured to the front panel by a fastener and hinges along a bottom edge of the bag to define the bottom recloseable opening into the bag interior.

In another embodiment, the bag bottom is releasably secured to the rear panel by a fastener and hinges along a bottom edge of the bag to define the bottom recloseable opening into the bag interior.

In another embodiment, the stoma opening includes a circular opening located on the rear panel.

In another embodiment, the stoma opening includes a slit extending from a top edge of the bag and at least partially towards a bottom edge of the bag.

In another embodiment, the stoma opening includes a circular opening and a slit located at at least one of above or below the circular opening.

In another embodiment, the case further includes at least one exterior pocket secured onto an exterior of the front panel, the at least one exterior pocket having a closure.

In another embodiment, the exterior pocket hinges to along a bottom edge of the exterior pocket to define a front recloseable opening.

In another embodiment, the exterior pocket includes at least one of at least one interior pocket and at least one slit.

### BRIEF DESCRIPTION OF THE DRAWINGS

FIG. 1 is a front view of an exemplary waist supported ostomy appliance case according to the disclosure.

FIG. 2 is a rear view of the case of FIG. 1, wherein a rear wall includes a stoma opening including an approximately circular opening.

FIG. 3 is a front view of the case of FIG. 1, wherein a bag top is lifted away from a rear panel to define a top recloseable opening and a bag bottom is pulled away from a front panel to define a bottom recloseable opening.

FIG. 4 is front view of an embodiment of the case of FIG. 1, wherein a bag top is releasable secured to a front panel and a bag bottom is releasaby secured to a rear panel.

FIG. 5 is a front view of the case of FIG. 4, wherein the bag top is lifted away from the front panel to define a top recloseable opening and the bag bottom is pulled away from the rear panel to define a bottom recloseable opening.

FIG. 6 is a rear view of an embodiment of the case of FIG. 1, wherein a stoma opening includes a slit extending from a top edge of a bag to substantially a bottom edge of a bag.

FIG. 7 is a rear view of an embodiment of the case of FIG. 1, wherein a stoma opening includes a slit extending from a top edge of a bag to substantially a midpoint in a rear panel.

FIG. 8 is a rear view of an embodiment of the case of FIG. 1, wherein a stoma opening includes a combination of a circular opening and a slit.

FIG. 9 is a front view of an embodiment of the case of FIG. 1, wherein the case includes an exterior pocket.

FIG. 10 is a front view of the case of FIG. 10, wherein the exterior pocket is pulled away from a front panel to define a front recloseable opening.

### DETAILED DESCRIPTION

In the description that follows, like components have been given the same reference numerals, regardless of whether they are shown in different embodiments. To illustrate an embodiment(s) of the present disclosure in a clear and concise manner, the drawings may not necessarily be to scale and certain features may be shown in somewhat schematic form. Features that are described and/or illustrated with respect to one embodiment may be used in the same way or in a similar way in one or more other embodiments and/or in combination with or instead of the features of the other embodiments.

The present disclosure has particular application to a case for concealing an ostomy appliance such as an ostomy bag or pouch. Of course, the principles of the disclosure may be useful for any medical appliance where is it desirable to conceal bodily fluids and allow for easy drainage thereof.

An exemplary waist supported ostomy appliance case includes a belt for securing the case to the waist of an individual and a bag positioned on the belt for enclosing an ostomy appliance. The bag includes a bag interior defined between a rear panel, a front panel joined to the rear panel at opposite sides of the bag, a bag top, and a bag bottom. The rear panel includes a stoma opening configured to fit around a flange of the ostomy appliance. The bag bottom includes a bottom recloseable opening into the bag interior. The bottom recloseable opening provides access to a bottom portion of the ostomy appliance. The case may include an exterior pocket secured onto an exterior of the front panel for holding personal belongings.

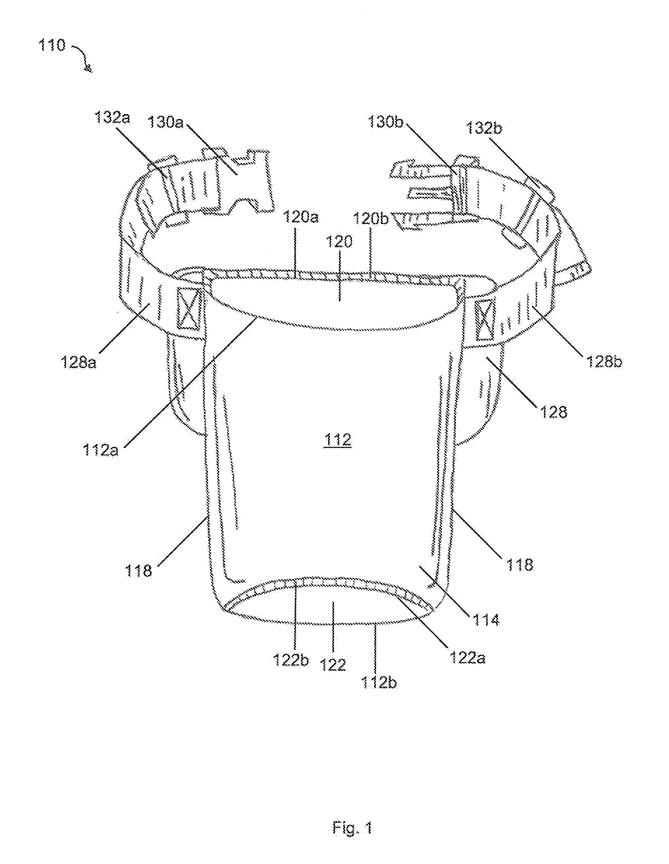

Referring now in detail to the drawings, and initially to FIGS. 1-3, a waist supported ostomy appliance case is shown at 110. The depicted case 110 includes a bag 112 of generally elongated shape having a front panel 114 and a rear panel 116 joined at two opposite side seams 118, a bag top 120, and a bag bottom 122.

The front panel 114, rear panel 116, bag top 120, and bag bottom 122 may be a single layer or multiple layers of fabric. The fabric may be knitted, woven, nonwoven, a polymer sheer, or a mixture of two or more thereof. The fabric may include cotton, wool, linen, silk, polyester, nylon, rayon, acrylics, polyethylene, polypropylenes, vinyls, acetates, acrylates, or a mixture of two or more thereof. The fabric may include any suitable cloth, such as so called “ballistic” nylon fabric conventionally used in luggage generally and fanny packs in particular. The seams 118 of the case 110 may be edged with bias tape for the further comfort of the individual wearing the case. Since the rear panel 116 faces and contacts the individual, it may be preferable that the rear panel 116 of the case be made of materials that are skin friendly (i.e., promote skin health, or at least do not negatively affect skin health).

A bag interior 124 is defined between the front panel 114 and the rear panel 116 and is sized to accommodate an ostomy appliance 126. The bag interior 124 may be any size to accommodate the variety of ostomy appliances in use. The bag interior 124 may have a length in the range of about 10 cm to 50 cm, or in another embodiment, in the range of about 10 cm to 40 cm, or in another embodiment, in the range of about 10 cm to 30 cm, or in another embodiment, in the range of about 10 cm to 25 cm, or in another embodiment, in the range of about 10 cm to 20 cm. The bag interior 124 may have a width in the range of about 5 cm to 25 cm, or in another embodiment, in the range of about 5 cm to 15 cm, or in another embodiment, in the range of about 5 cm to 10 cm.

A belt 128 includes a first belt member 128a and a second belt member 128b each attached to a corresponding one of the bag's 112 opposite side seams 118. The first belt member 128a and the second belt member 128b are provided with cooperating fasteners 130a, 130b for releasably fastening the case 110 around an individual's waist, with the bag 112 positioned over the individual's abdomen. The cooperating fasteners 130a, 130b may be, for example, a snap buckle, a hook and loop fastener, a button, a slide fastener, a key ring, a clamping or holding device, a pin and hole buckle, or an equivalent thereof.

Adjustment of the first belt member 128a to fit individuals having various waist sizes is achieved by providing a releasable locking clasp 132a on the first belt member 128a. The locking clasp 132a permits an individual to draw the first belt member 128a therethrough and lock the fastener 130a at a specific location on the first belt member 128a. In addition, the second belt member 128b is provided with a locking clasp 132b permitting the adjustment of the length of the second belt member 128b. The locking clasp 132b permits an individual to draw the second belt member 128b therethrough and lock the fastener 130b at a specific location on the second belt member 128b. The locking clasps 132a, 132b of the first and second belt members 128a, 128b permit an individual to adjust the length of the first and second belt members 128a, 128b so that the waist supported ostomy appliance case 110 fits comfortably about the waist of the individual.

The bag top 120 is attached to the front panel 114 along top edge 112a of bag 112. The rear edge 120a of the bag top 120 is releasably secured to the rear panel 116 by a fastener 120b such as a zipper, hook and loop fastener, button, or snap. The bag top 120 hinges along top edge 112a and when lifted away from the rear panel 116 defines a top recloseable opening 120c into the bag interior 124 for insertion of the ostomy appliance 126 into the bag interior 124.

The bag bottom 122 is attached to the rear panel 116 along bottom edge 112b of bag 112. The front edge 122a of the bag bottom 122 is releasably secured to the front panel 114 by a fastener 122b such as a zipper, hook and loop fastener, button, or snap. The bag bottom 122 hinges along the bottom edge 112b and when pulled away from the front panel 114 defines a bottom reclosable opening 122c into the bag interior 124 to aid the user in draining the ostomy appliance 126. The bottom reclosable opening 122c allows the individual to access a bottom portion 126a of the ostomy appliance 126, such as a drain tube of a urostomy bag or a throat at the bottom of an ilestomy pouch, so that the ostomy appliance 126 may be emptied without removing the case 110.

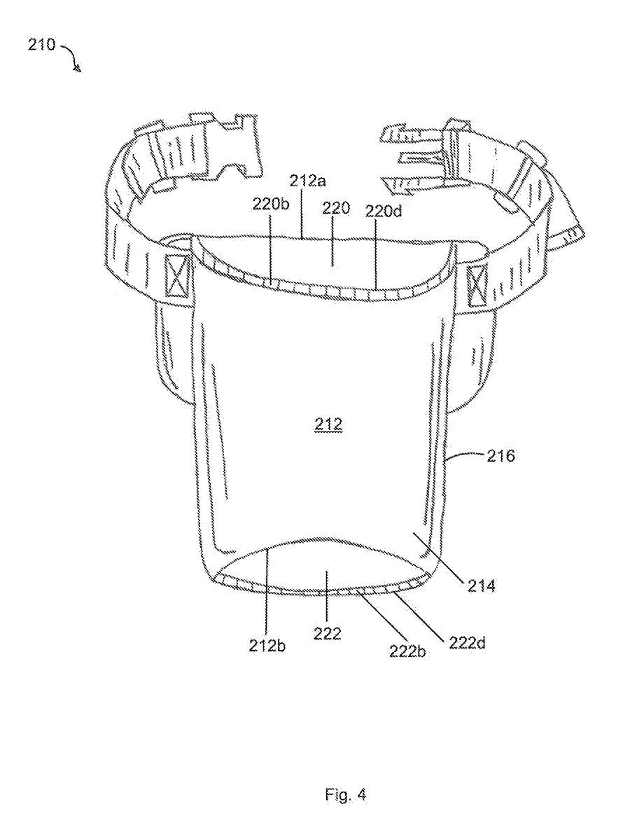

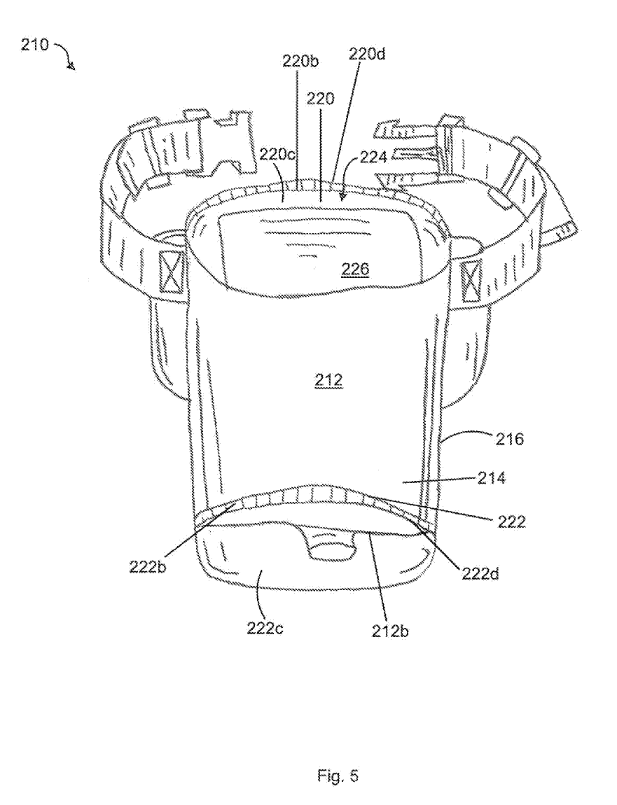

Referring now to FIGS. 4 and 5, in another embodiment, a front edge 220d of a bag top 220 of a case 210 is releasably secured to a front panel 214 by a fastener 220b such as a zipper, hook and loop fastener, button, or snap. The bag top 220 hinges along top edge 212a of bag 212 and when lifted away from the front 214 panel defines a top recloseable opening 220c into a bag interior 224 for insertion of an ostomy appliance 226 into the bag interior 224.

In another embodiment, the rear edge 222d of the bag bottom 222 of case 210 is releasably secured to the rear panel 216 by a fastener 222b such as a zipper, hook and loop fastener, button, or snap. The bag bottom 222 hinges along bottom edge 212b of bag 212 and when pulled away from the rear panel 216 defines a bottom reclosable opening 222c into the bag interior 224 to aid the user in draining the ostomy appliance 226.

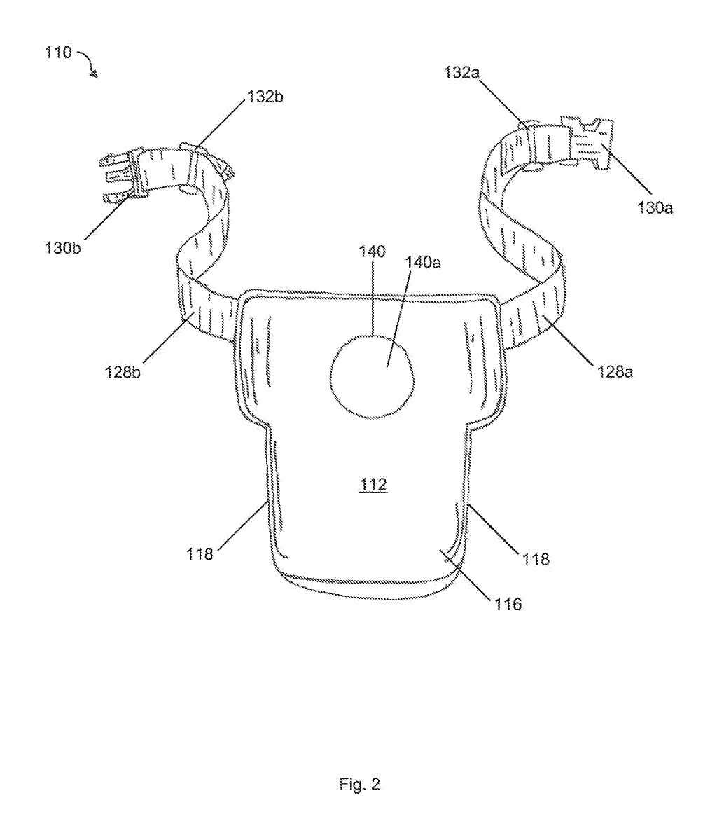

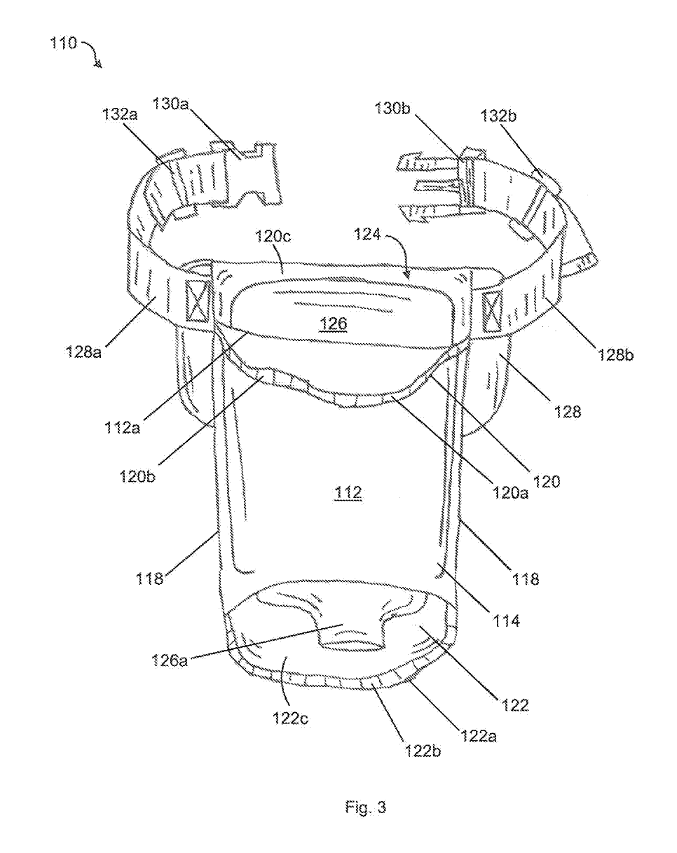

The ostomy appliance may include a skin barrier and a flange onto which the ostomy appliance connects to the stoma. Referring back to FIG. 2, the rear panel 116 includes a stoma opening 140 that fits around a stoma and a flange on a skin barrier of the individual. The stoma opening 140 may include an approximately circular opening 140a that fits around the stoma and the flange on the skin barrier. The circular opening 140a is located on the rear panel 116 such that the individual may insert the ostomy appliance 126 (FIG. 3) through the top reclosable opening 120c (FIG. 3), secure the appliance 126 to the flange, and comfortably secure the case thereto. The circular opening 140a may have a diameter in the range of 1 cm to 10 cm, or in another embodiment, in the range of 1 cm to 5 cm, or in another embodiment, in the range of 5 cm to 10 cm.

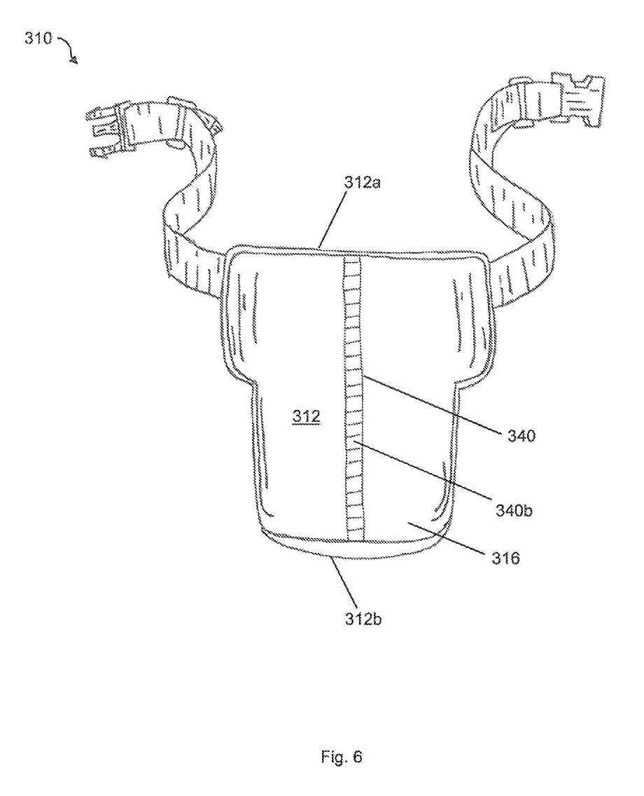

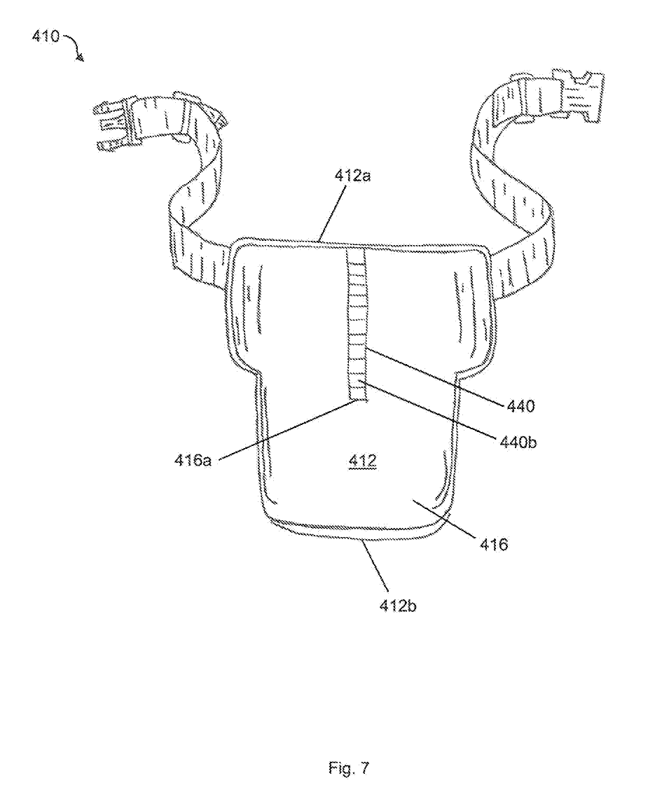

Referring now to FIGS. 6 and 7, in another embodiment, the stoma opening 340, 440 of cases 310, 410 may include a slit 340b, 440b extending from a top edge 312a, 412a of a bag 312, 412 and at least partially towards a bottom edge 312b, 412b of the bag 312, 412. The slit may include a fastener such as a zipper, hook and loop fastener, button, or snap to close the rear panel 316, 416 around the flange. As shown in FIG. 6, the slit 340b of the stoma opening 340 may extend from top edge 312a to substantially the bottom edge 312b. This configuration allows the individual to insert the ostomy appliance 126 (FIG. 3) through the rear panel 316 with or without removal of the ostomy appliance 126 from the flange and secure the case thereto. This configuration also allows an individual, when the bag top 120 (FIG. 1) is lifted away from the rear panel 216, to slide the case around the flange with or without removal of the ostomy appliance 126 from the stoma, close the bag top 120, and secure the case thereto. As shown in FIG. 7, the slit 440b of stoma opening 440 may extend from top edge 412a to substantially a midpoint 416a in the rear panel 416. When the bag top 120 is lifted away from the rear panel 416, this configuration allows the individual to slide the case around the flange with or without removal of the ostomy appliance 126 from the stoma, close the bag top 120, and secure the case thereto.

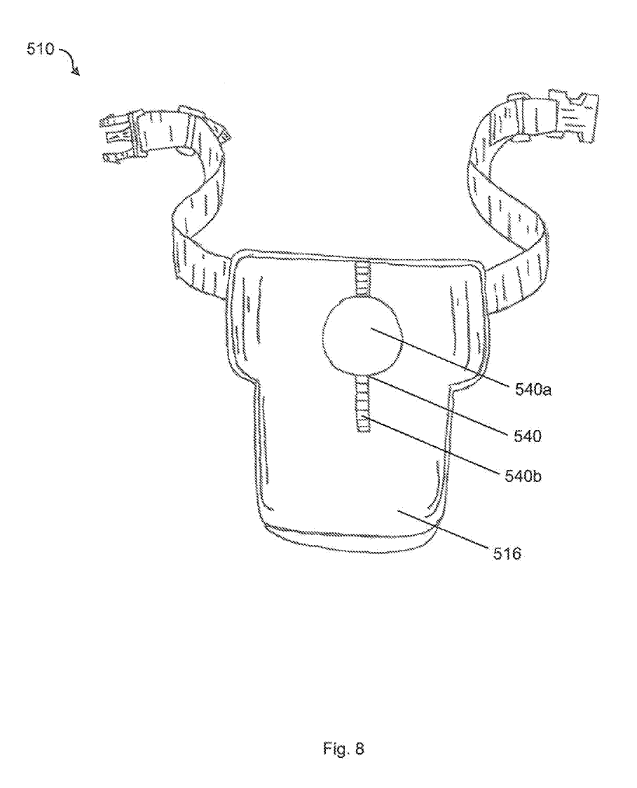

Referring now to FIG. 8, in another embodiment, the stoma opening 540 of case 510 may include a combination of a circular opening 540a and a slit 540b with a fastener. The circular opening 540a is located on the rear panel 516 such that the individual may insert the ostomy appliance 126 (FIG. 3) through the top reclosable opening 120c (FIG. 3), secure the appliance 126 to the flange, and comfortably secure the case thereto. The slit 540b is located at at least one of above or below the circular opening 540a and allows the individual to close the rear panel 516 around the flange.

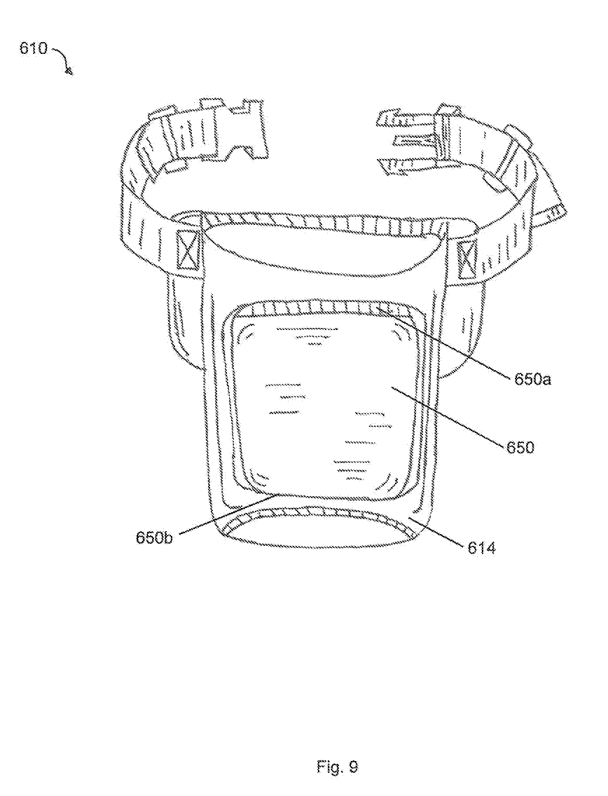

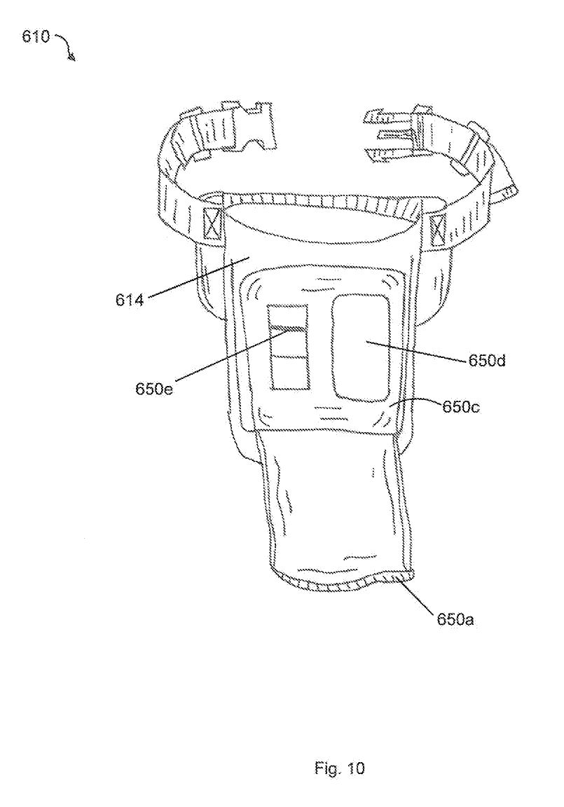

The case serves the dual functions of enclosing and supporting the ostomy appliance and as a carrying case or pouch. Pockets may be provided on the exterior of the case. For example, referring to FIGS. 9 and 10, the case 610 may include at least one exterior pocket 650 secured onto an exterior of the front panel 614 and provided with a closure 650a such as a zipper, hook and loop fastener, button, or snap. The exterior pocket 650 hinges along a bottom edge 650b of the exterior pocket 650 and when pulled away from the front panel 614 defines a front recloseable opening 650c. The exterior pocket 650 may include at least one interior pocket 650d and/or at least one slit 650e to secure personal items such as money, credit cards, and makeup.

The present disclosure provides a waist supported ostomy appliance case 110 that effectively and comfortably conceals an ostomy appliance 126. The case 110 includes a belt 128 for securing the case 110 to the waist of an individual and a bag 112 positioned on the belt 128 for enclosing the ostomy appliance 126. A rear panel 116 of the bag 112 includes a stoma opening 140 configured to fit around a flange of the ostomy appliance 126. A bag bottom 122 of the bag 112 includes a bottom recloseable opening 122c into a bag interior 124. The bottom recloseable opening 122c provides access to a bottom portion 126a of the ostomy appliance 126 for emptying the ostomy appliance 126 without the need to remove the case 110. The case 110 may include an exterior pocket 650 for storing personal belongings.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated to embodiments, such feature may be combined with one or more other features of the other embodiments, as may be desired and advantageous for any given or particular application.

What is claimed is:

1. A waist supported ostomy appliance case, comprising:
a belt for securing the case to the waist of an individual; and
a bag positioned on the belt for enclosing an ostomy appliance, the bag including a bag interior defined between a rear panel, a front panel joined to the rear panel at opposite sides of the bag, a bag top, and a bag bottom,
wherein the rear panel includes a stoma opening configured to fit around a flange of the ostomy appliance, and
wherein the bag bottom includes a bottom recloseable opening into the bag interior, the bottom recloseable opening providing access to a bottom portion of the ostomy appliance.

2. The case of claim 1, wherein the belt includes a first belt member and a second belt member each attached to a corresponding one of the opposite sides of the bag.

3. The case of claim 2, wherein the first belt member and the second belt member include cooperating fasteners.

4. The case of claim 3, wherein at least one of the first belt member and the second belt member includes a locking clasp.

5. The case of claim 1, wherein the bag top is releasably secured to the rear panel by a fastener and hinges along a top edge of the bag to define a top recloseable opening into the bag interior providing access for insertion of the ostomy appliance into the bag interior.

6. The case of claim 1, wherein the bag top is releasably secured to the front panel by a fastener and hinges along a top edge of the bag to define a top recloseable opening into the bag interior providing access for insertion of the ostomy appliance into the bag interior.

7. The case of claim 1, wherein the bag bottom is releasably secured to the front panel by a fastener and hinges along a bottom edge of the bag to define the bottom recloseable opening into the bag interior.

8. The case of claim 1, wherein the bag bottom is releasably secured to the rear panel by a fastener and hinges along a bottom edge of the bag to define the bottom recloseable opening into the bag interior.

9. The case of claim 1, wherein the stoma opening includes a circular opening located on the rear panel.

10. The case of claim 1, wherein the stoma opening includes a slit extending from a top edge of the bag and at least partially towards a bottom edge of the bag.

11. The case of claim 1, wherein the stoma opening includes a circular opening and a slit located at at least one of above or below the circular opening.

12. The case of claim 1, wherein the case further includes at least one exterior pocket secured onto an exterior of the front panel, the at least one exterior pocket having a closure.

13. The case of claim 12, wherein the exterior pocket hinges along a bottom edge of the exterior pocket to define a front recloseable opening.

14. The case of claim 12, wherein the exterior pocket includes at least one of at least one interior pocket and at least one slit.
